(19) United States
(12) Patent Application Publication
(10) Pub. No.: US 2016/0367396 A1
(43) Pub. Date: Dec. 22, 2016

(54) COLD CAP

(71) Applicant: Oronzo Triggiano, Atlanta, GA (US)

(72) Inventor: Oronzo Triggiano, Atlanta, GA (US)

(21) Appl. No.: 14/743,083

(22) Filed: Jun. 18, 2015

(51) Int. Cl. A61F 7/10

(52) U.S. Cl. CPC A61F 7/10; A61F 2007/0008

(57) ABSTRACT

Implementations of a cold cap are provided. In some implementations, the cold cap may be worn on the head by cancer patients during chemotherapy treatments to prevent hair loss on their scalp. In some implementations, the cold cap may comprise a central portion, a back portion, a first side portion, a second side portion, a chin strap, and a compression strap. In some implementations, the cold cap is configured to fold around a person's head when being worn. In some implementations, the central portion, back portion, first and second side portions, and compression strap each have an interior compartment therein configured to contain a cold retaining medium. In some implementations, the compression strap may be used to compress the back portion of the cold cap against the scalp of the wearer. In this way, hair loss on the backside of the wearer's head may be minimized or prevented.

### TECHNICAL FIELD

This disclosure relates to implementations of a cold cap.

### BACKGROUND

Traditional cytotoxic chemotherapeutic agents used during chemotherapy are known to kill cells that rapidly divide, a well known property of most cancer cells. As a result, chemotherapeutic agents also kill cells that rapidly divide under normal circumstances such as cells in the bone marrow, intestines, and hair follicles. Hair loss as a result of chemotherapy treatment(s) is a well known side effect. It is also known that cooling the scalp of a patient during their chemotherapy treatment(s) may reduce or eliminate hair loss that normally results from exposing the hair follicles to cytotoxic chemotherapeutic agents.

U.S. Pat. No. 5,897,581 (“'581 patent”) discloses headwear for use in applying cold to a person's scalp. The headwear has double skinned portions that contain a cold retaining medium configured to cool the scalp of a wearer. However, the headwear disclosed in the '581 patent has several disadvantages. First, the headwear is not configured to cool the entire portion of the scalp on the backside of a wearer's head. Second, the '581 patent does not provide a means to compress the backside of the headwear against the scalp on the backside of a wearer's head.

### BRIEF DESCRIPTION OF THE DRAWINGS

FIG. 1 illustrates a view of the underside of an example implementation of the cold cap according to the principles of the present disclosure.

FIG. 2 illustrates a compression strap according to the principles of the present disclosure.

FIGS. 3A-3C illustrate an example use of the cold cap and compression strap shown in FIGS. 1 and 2.

### DETAILED DESCRIPTION

Implementations of a cold cap are provided. In some implementations, the cold cap may be worn on the head by cancer patients during chemotherapy treatments to prevent hair loss on their scalp. In some implementations, the cold cap may be used to cool the scalp of the wearer thereby reducing blood flow to the hair follicles of the scalp. By reducing the blood flow to the hair follicles, the hair follicles may receive diminished concentrations of cytotoxic chemotherapeutic agents during treatment. In this way, hair loss on the scalp may be minimized or prevented by the cold cap disclosed herein.

In some implementations, the cold cap may comprise a central portion, a back portion, a first side portion, a second side portion, and a chin strap. In some implementations, the cold cap may further comprise a compression strap. In some implementations, the compression strap may be used to compress the back portion of the cold cap against the scalp of the wearer. In this way, hair loss on the backside of the wearer's head may be minimized or prevented. In some implementations, the compression strap may have the general shape of a chevron.

In some implementations, the central portion, back portion, first and second side portions, and compression strap each have an interior compartment therein configured to contain a cold retaining medium.

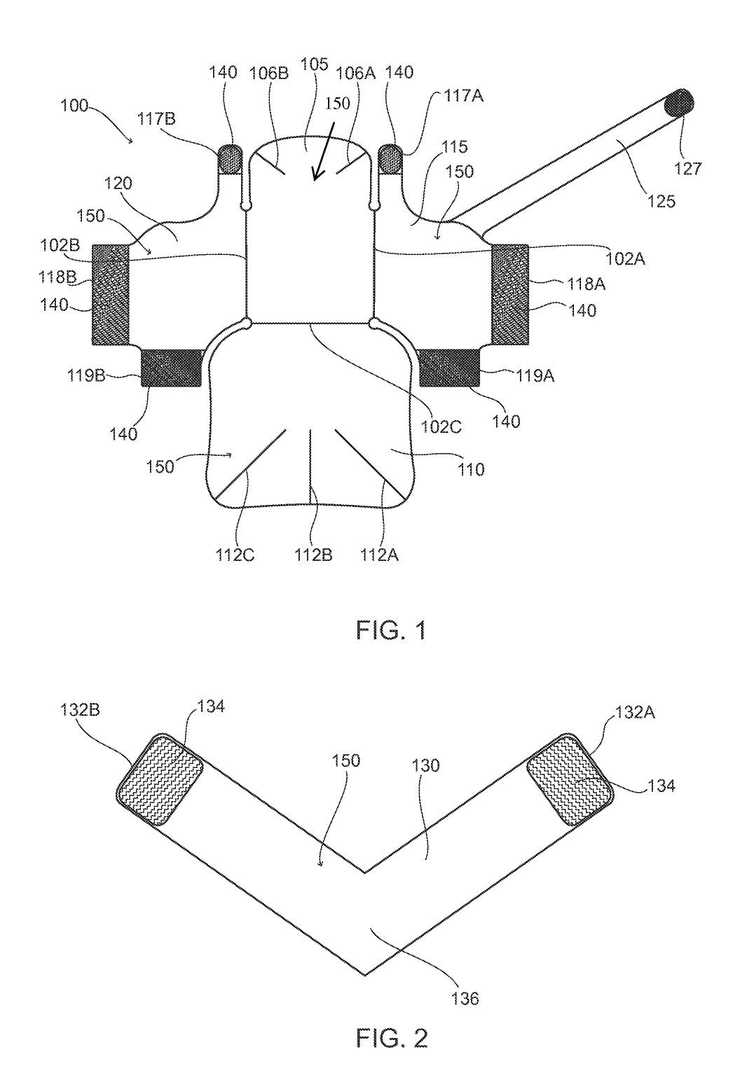

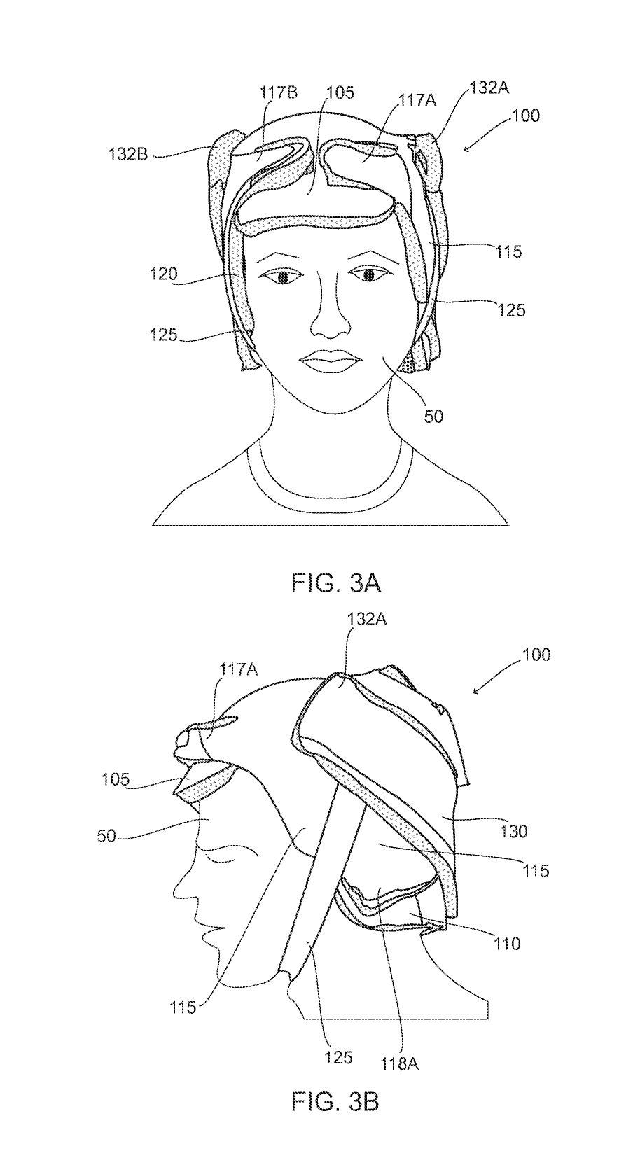

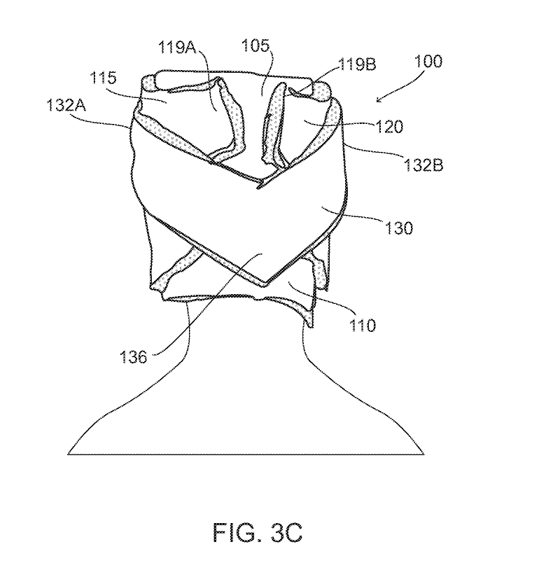

FIGS. 1 and 2 illustrate an example implementation of a cold cap 100 according to the principles of the present disclosure. In some implementations, the cold cap 100 is configured to fold around a person's head 50 when being worn (see, e.g., FIGS. 3A-3C). In some implementations, the cold cap 100 may lie flat when not being worn (see, e.g., FIG. 1).

In some implementations, as shown in FIG. 1, the cold cap 100 may comprise a central portion 105, a back portion 110, a first side portion 115, a second side portion 120, and a chin strap 125. In some implementations, as shown in FIG. 2, the cold cap 100 may further comprise a compression strap 130. In some implementations, the compression strap 130 may be used to compress the back portion 110 of the cold cap 100 against the scalp of the wearer. In this way, hair loss on the backside of the wearer's head may be minimized or prevented.

In some implementations, as shown in FIG. 3A, the central portion 105 of the cold cap 100 may be configured to cover at least a portion of the scalp of the top and/or forehead of a wearer's head 50. In some implementations, the first side portion 115, the second side portion 120, and the back portion 110 extend from the central portion 105 of the cold cap 105 (see, e.g., FIG. 1). In some implementations, a seam 102A, 102B, 103C (collectively 102) separates the central portion 105 from the first side portion 115, the second side portion 120, and the back portion 110 of the cold cap 100, respectively (see, e.g., FIG. 1). In this way, through the positioning of the seams 102, the cold cap 100 may be flexible.

As shown in FIG. 1, in some implementations, a front end of the central portion 105 may have a first seam 106A and a second seam 106B (collectively 106) thereon. In some implementations, the two seams 106 extend from a side edge of the central portion 105 at an angle (see, e.g., FIG. 1). In this way, through the positioning of the seams 106, the front end of the central portion 105 may be flexible.

In some implementations, as shown in FIG. 3A, the first side portion 115 and the second side portion 120 of the cold cap 100 may be configured to cover at least a portion of the scalp of the left side and the right side, respectively, of a wearer's head 50. In some implementations, the first and second side portions 115, 120 may each have extending therefrom a first flap 117A, 117B (collectively 117), a second flap 118A, 118B (collectively 118), and/or a third flap 119A, 119B (collectively 119), respectively (see, e.g., FIG. 1). In some implementations, the first flap 117, the second flap 118, and/or the third flap 119 of the first and second side portions 115, 120 may each include a fastener 140 thereon (see, e.g., FIG. 1).

In some implementations, as shown in FIG. 1, the chin strap 125 may extend from a side edge of the first side portion 115. In some implementations, the chin strap 125 may extend from a side edge of the second side portion 120 (not shown). In some implementations, the chin strap 125 may include a fastener 127 on a distal end thereof (see, e.g., FIG. 1). In some implementations, the fastener 127 on the distal end of the chin strap 125 may be used to secure the chin strap 125 underneath the jaw of a wearer (see, e.g., FIGS. 3A and 3B). In this way, the chin strap 125 may be used to assist with securing the cold cap 100 in position on the head 50 of a wearer. In some implementations, the fastener 127 on the chin strap 125 may be another fastening means such as snaps, buttons, magnets, other reclosable fasteners, or any other attachment or fastening technology existing or developed in the future to secure the distal end of the chin strap 125 to the cold cap 100.

In some implementations, as shown in FIG. 3C, the back portion 110 of the cold cap 100 may be configured to cover at least a portion of the wearer's scalp on the backside of the head 50. In some implementations, a back end of the back portion 110 may have a first seam 112A, a second seam 112B, and a third seam 112C (collectively 112) thereon. In some implementations, each seam 112 may extend from a side edge of the back portion 110 (see, e.g., FIG. 1). In some implementations, through the positioning of the three seams 112, the back end of the back portion 105 may be flexible.

As shown in FIG. 2, the compression strap 130 may have the general shape of a “V” or an inverted chevron. In some implementations, the tip or point of a chevron or “V” shaped compression strap 130 may be rounded. In some implementations, the compression strap 130 may be any suitable shape. In some implementations, the compression strap 130 may have a first end 132A, a second end 132B (collectively 132), and a middle portion 136. In some implementations, the compression strap 130 may include a fastener 134 on each end 132 (see, e.g., FIG. 2). As shown in FIG. 3, in some implementations, a middle portion 136 of the compression strap 130 may be fixedly secured to the back portion 110 of the cold cap 100. In some implementations, the middle portion 136 of the compression strap may be removably secured to the back portion 110 of the cold cap 100 (not shown). In some implementations, the fasteners 134 may be other fastening means such as snaps, buttons, magnets, other reclosable fasteners, or any other attachment or fastening technology existing or developed in the future to secure the ends 132 of the compression strap 130 to the cold cap 100.

In some implementations, the central portion 105, back portion 110, first and second side portions 115, 120, and compression strap 130 each have an interior compartment 150 therein configured to contain a cold retaining medium. In some implementations, the interior compartment 150 of the central portion 105, back portion 110, first side portion 115, second side portion 120, and/or compression strap 130 is comprised of at least a first layer of material and a second layer of material. In some implementations, portions of the first and second layers of material are sealed together to form an interior compartment 150. In this way, the volume of the interior compartment 150 is created and the cold retaining medium sealed therein. In some implementations, the seals may be formed by radio frequency welding. In some implementations, the seals may be formed using any method known to one or ordinary skill in the art. In some implementations, the first and second layers of material may be polyurethane. In some implementations, the first and second layers of material may retain flexibility when cold. In some implementations, the first and second layers of material may be any suitable material that can withstand being cooled to at least a temperature of −38° C.

As shown in FIGS. 3A-3C, in some implementations, the fasteners 140 found on the first flaps 117, second flaps 118, and third flaps 119 of the first and second side portions 115, 120 may be used to secure the cold cap 100 about a wearer's head 50. In some implementations, the fasteners 140 may be hook and loop fasteners (e.g., Velcro®) (see, e.g., FIG. 1). In some implementations, the fasteners 140 may be other fastening means such as snaps, buttons, magnets, other reclosable fasteners, or any other attachment or fastening technology existing or developed in the future to secure the cold cap 100 about a wearer's head 50.

In some implementations, the cold cap 100 may include one or more insulating layers of material on its exterior to help prevent the loss of cold to the surrounding environment.

In some implementations, the cold retaining medium used with the cold cap 100 may be a gel. In some implementations, the cold retaining medium may be propylene glycol gel with additives such as glycerin and/or water. In some implementations, the cold retaining medium may be a liquid. In some implementations, the cold retaining medium may be any suitable chemical or chemical compound that can give up its cold.

In some implementations, the seams 102, 106, 112 may be formed by radio frequency welding. In some implementations, the seams 102, 106. 112 may be formed using any method known to one or ordinary skill in the art having the benefit of the present disclosure and in view of the material (e.g., synthetic, semi-synthetic, or natural material(s)) that the cold cap 100 is manufactured from.

As shown in FIGS. 3A-3C, to use the cold cap 100, the cold cap 100 may be initially positioned on a wearer's head 50 so that the central portion 105 is covering the scalp of the top of the wearer's head 50, the back portion 110 is covering the scalp on backside of the head 50, and the first and second side portions 115, 120 are covering the scalp on the left side and the right side, respectively, of the head 50.

Next, in some implementations, the chin strap 125 is pulled underneath the wearer's chin and secured by the fastener 127 thereon to the second side portion 120 of the cold cap 100 (see, e.g., FIG. 3A).

Then, in some implementations, the first flap 117A, 117B extending from the first and second side portions 115, 120, respectively, are each secured by their respective fasteners 140 to the exterior of the central portion 105 near the front end thereof (see, e.g., FIGS. 3A-3B).

Next, in some implementations, the second flap 118A, 118B extending from the first and second side portions 115, 120, respectively, are each secured by their respective fasteners 140 to the exterior of the back portion 110 near the back end thereof (see, e.g., FIG. 3B). Then, the third flap 119A, 119B of the first and second side portions 115, 120, respectively, are each secured by their respective fasteners 140 to the exterior of the back portion 110 near the seam 102C (see, e.g., FIG. 3C).

In some implementations, the compression strap 130 may be used to compress the back portion 110 of the cold cap 100 against the scalp of the wearer. In this way, there may be no gap between the scalp of the wearer and the back portion 110 of the cold cap 100.

Initially, in some implementations, the first end 132A of the compression strap 130 may be pulled towards the front end of the central portion 105 and secured to the exterior of the first side portion 115 of the cold cap 100 (see, e.g., FIG. 3A and 3B). In this way, the compression strap 130 may also be used to press the first side portion 115 against the wearer's scalp.

Then, in some implementations, the second end 132B of the compression strap 130 may be pulled towards the front end of the central portion 105 and secured to the exterior of the second side portion 120 of the cold cap 100 (see, e.g., FIG. 3A). In this way, the compression strap 130 may be used to press the second side portion 120 against the wearer's scalp.

In some implementations, after both ends 132 of the compression strap 130 are secured in place, the middle portion 136 presses the back portion 110 of the cold cap 100 against the wearer's scalp (see, e.g., FIG. 3C). In this way, hair loss on the back side of a wearer's head may be minimized or prevented.

In some implementations, the cold cap 100 may be cooled down to a temperature of at least −38° C. prior to use. In some implementations, the cold cap 100 may be cooled down to a temperature greater than −38° C. or less than −38° C. prior to use.

Reference throughout this specification to “an embodiment” or “an implementation” or words of similar import means that a particular described feature, structure, or characteristic is included in at least one embodiment of the present invention. Thus, the phrase “in an embodiment” or “an implementation” or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

1. A cold cap configured to be secured about the head of a wearer, the cold cap comprising:
a central portion having an interior compartment containing a cold retaining medium;
a first side portion having an interior compartment containing a cold retaining medium, the first side portion extending from a first side of the central portion;
a second side portion having an interior compartment containing a cold retaining medium, the second side portion extending from a second side of the central portion;
a back portion having an interior compartment containing a cold retaining medium, the back portion extending from a third side of the central portion; and
a compression strap having a first end, a second end, a middle portion, and an interior compartment containing a cold retaining medium, the middle portion of the compression strap is secured to an exterior side of the back portion and the first end and the second end each have a fastener thereon.

2. The cold cap of claim 1, wherein the compression strap has the shape of a “V”.

3. The cold cap of claim 1, wherein the first side, the second side, and the third side of the central portion is a flexible seam.

4. The cold cap of claim 1, further comprising a chin strap extending from a side edge of the first side portion.

5. The cold cap of claim 1, further comprising a chin strap extending from a side edge of the second side portion.

6. The cold cap of claim 1, wherein the cold retaining medium is comprised of propylene glycol, glycerin, and water.

7. A method of using the cold cap of claim 1, the method comprising:
positioning the cold cap on the head of a wearer;
securing the first side portion to an exterior side of the central portion and an exterior side of the back portion;
securing the second side portion to the exterior side of the central portion and the exterior side of the back portion;
pulling the first end of the compression strap towards a front end of the central portion and securing it to the exterior side thereof; and
pulling the second end of the compression strap towards the front end of the central portion and securing it to the exterior side thereof.

8. The method of claim 7, further comprising:
extending a portion of the compression strap that lies between the middle portion and the first end thereof across the first side portion of the cold cap thereby pressing the first side portion against the head of the wearer; and
extending a portion of the compression strap that lies between the middle portion and the second end thereof across the second side portion of the cold cap thereby pressing the second side portion against the head of the wearer.
